(19) United States
(12) Patent Application Publication
(10) Pub. No.: US 2026/0357231 A1
(43) Pub. Date: Nov. 12, 2026

(54) MODULAR CRISPR SYSTEM

(71) Applicant: SOLEDITS AB, Lund (SE)

(72) Inventors: Per HOFVANDER, BJÄRRED (SE); Mariette ANDERSSON, LUND (SE); Niklas OLSSON, TORNA HÄLLESTAD (SE); Martin FRIBERG, ARLÖV (SE); Paul VOGEL, MALMÖ (SE)

(73) Assignee: SOLEDITS AB, Lund (SE)

(21) Appl. No.: 19/125,518

(22) PCT Filed: Oct. 31, 2023
(86) PCT No.: PCT/SE2023/051082

§ 371 Date: Apr. 29, 2025

(30) Foreign Application Priority Data
Oct. 31, 2022 (SE) 2251269-3

(51) Int. Cl. C12N 9/22; C12N 9/10; C12N 9/12; C12N 9/78; C12N 15/10; C12N 15/113; C12N 15/82

(52) U.S. Cl. CPC C12N 9/226; C12N 9/1007; C12N 9/1276; C12N 9/78; C12N 15/102; C12N 15/113; C12N 15/8202; C12N 15/8213; C12Y 207/07049; C12N 2310/20

(57) ABSTRACT

A modular CRISPR-Cas system for ribonucleoprotein (RNP) delivery is provided, the CRISPR-Cas system comprising a CRISPR-Cas protein fused to a first binding peptide, and a functional protein fused to a second binding peptide, wherein the first binding peptide and the second binding peptide are configured to bind to each other and define a binding peptide pair. A method of assembling a CRISPR-Cas-complex for ribonucleoprotein (RNP) delivery and a CRISPR-Cas complex, as well as the use of the modular CRISPR-Cas system or the CRISPR-Cas complex for modifying the genome of an organism, is also provided.

### TECHNICAL FIELD

The technology proposed herein generally relates to the field of genome editing using CRISPR-Cas systems.

### BACKGROUND

DNA is a molecule that stores genetic information and functions as a blueprint for any living organism on earth. Manipulation of the genetic information can alter the organism. This has been indirectly done by humans to breed crop species with beneficial properties such as higher yield or better robustness. Plants that showed a preferred characteristic were selected for breeding. In this way plants with superior traits were created.

Climate change and the growing human population already pose severe challenges for food security and agricultural businesses around the globe. One key component on a way to a more sustainable agriculture that has the ability to adapt to changing environmental conditions could be the newly arisen genome editing. A form of genetic engineering that allows for precise edits without introducing foreign DNAs into the target organism. The core of genome editing is the gene scissor CRISPR-Cas that can be programmed to edit at a specific position in the genome.

CRISPR-Cas systems introduce DSBs at specific positions, but the repair through non-homologous end-joining (NHEJ) is rather random and screening for the desired mutation can be laborious. Recent advancements enable more precise edits and changes. Mainly, two different approaches are in use: Base editors (BE) and prime editors (PE). Both systems involve fusing a functional protein to a variant of Cas9.

First generation BEs use a catalytically dead Cas9 (dCas9), a variant in which both cleavage domains are inactivated by mutations but the RNA-programmable binding of DNA is retained. dCas9 is then fused to a cytidine deaminase enzyme. Deamination of cytidine (C) results in uracil (U), which is recognized by DNA repair mechanisms and replaced with thymine (T). Thus, this method converts a C:G base pair into A:T without introducing DSBs and is therefore called cytosine base editor (CBE).

Third generation CBEs achieved higher editing efficiency with a Cas9 nickase (nCas9) in which only the RuvC domain is inactivated. All four nucleotide conversions were made possible with the development of adenine base editors (ABE) by fusing nCas9 to an adenosine deaminase, which was obtained through extensive protein engineering.

PE offers even higher versatility. Here, an nCas9 is fused to the murine leukemia virus (MMLV)-reverse transcriptase (RT). The programmable RNA has a 3′ extension with a primer binding site (PBS) and is called prime editing guide RNA (pegRNA). In this system, nCas9 dislocates the target strand through binding to the 5′ end of the pegRNA. After that, the non-target strand is cleaved by the RuvC nuclease domain of nCas9 introducing a nick in the DNA. The PBS can now hybridize to the free 3′ end of the target DNA, acting as a primer for the RT, which reverse transcribe RNA to incorporate the desired sequence into the target DNA. Any sequence following the PBS of the pegRNA can thus be inserted into the DNA, which creates a powerful “search and replace” tool. Engineering of the MMLV-RT, nicking the non-edited strand and including the PAM sequence into the edit yielded PE systems with significantly increased efficiency. Theoretically, any deletion up to 44 bp and insertion of up to 80 bp can be achieved. So far over 50 BEs and PEs have been developed, of which each has very specific advancements and a different scope of application.

Depending on the application, a CRISPR-Cas system can be delivered to a cell in different forms: as DNA, as mRNA, and as a protein complex. Further, DNA can be transfected in a transient or stable manner. The former refers to the introduction of the CRISPR-Cas system as DNA via a plasmid vector that does not integrate into the genomic DNA of the cell. The plasmid DNA can be delivered for instance by the means of a gene gun or PEG transformation. For transient transformation the plasmid is usually lost after cell divisions. In contrast, stable integration of transgenes results in continuous expression that is maintained through generations. Some of the most common techniques for stable integration of transgenes into plant genomes are biolistics or Agrobacterium-mediated transformation. Stably transformed plants can be made transgene-free through self-pollination or crossing where only the desired mutation remains in the genome.

However, in both cases recombinant DNA is introduced into the cell, which might pose a risk for unpredicted side-effects. Delivery of mRNA encoding CRISPR-Cas enables faster expression and bypasses the risk of insertional mutagenesis. Because RNA is highly unstable and quickly degraded, delivery in the form of mRNA is however always transient.

Lastly, CRISPR-Cas delivery in protein form allows for instantaneous CRISPR activity and avoids the risk of introducing recombinant DNA into the cell. Specifically, prior to transfection, a CRISPR-Cas protein is loaded with guide RNA (gRNA) and then delivered to the cell as ribonucleoprotein (RNP). Means of delivery in plants include for instance biolistics, that allow for cell wall penetration, microinjection during sexual reproduction and protoplast transfection. The main steps of the workflow for the latter one include: isolation of protoplasts from plant tissue, RNP assembly and delivery to the protoplasts, which are subsequently regenerated to whole plants. This transgene-free method is advantageous because unintended effects from recombinant DNA is preserved.

Although RNP delivery has been successfully developed in several plant species using standard CRISPR-Cas techniques, applying the more advanced BE and PE approaches in RNP delivery remains a challenge. This is mainly due to the large size of the BE and PE fusion proteins, which makes it difficult, time-consuming and expensive, to produce large amounts of high quality protein in bacterial systems. Successful attempts have been made to produce the fusion proteins in mammalian cells, however, eukaryotic protein expression is much more resource intensive. As an example Rees et al., 2017 successfully produced BE fusion proteins in E. coli only after extensive optimization steps including testing a variety of different expression strains and constructs to achieve sufficient yield. Laborious optimization steps would therefor limit the application area.

Accordingly, there is a need for further developing and implementing base editing and prime editing with CRISPR-Cas for RNP delivery.

WO2021165508 discloses an expression cassette comprising a Type II or V CRISPR-Cas effector protein domain fused to a peptide tag and a reverse transcriptase domain fused to an affinity peptide.

US20200017869 discloses a recombinant vector comprising a first nucleic acid sequence comprising a plant promoter and that encodes a recombinant polypeptide comprising a nuclease-deficient CAS9 polypeptide (dCAS9) or fragment thereof and a multimerized epitope; a second nucleic acid sequence comprising a plant promoter and that encodes a recombinant polypeptide comprising a transcriptional activator and an affinity polypeptide that specifically binds to the epitope; and a third nucleic acid sequence comprising a promoter and that encodes a crRNA and a tracrRNA, or fusions thereof.

### OBJECTS OF THE TECHNOLOGY PROPOSED HEREIN

It is a first object of the technology proposed herein to provide a modular CRISPR-Cas system that allows for faster and simpler genome modification using base editing (BE) and prime editing (PE) or for homology directed repair (HDR), in particular using ribonucleoprotein delivery.

It is a second object of the technology proposed herein to provide method of assembling a CRISPR-Cas-complex.

It is a third object of the technology proposed herein to provide a CRISPR-Cas-complex.

It is a fourth object of the technology proposed herein to provide a use of a modular CRISPR-Cas system or a CRISPR-Cas-complex for modifying the genome of an organism.

### SUMMARY

At least one of the abovementioned objects, or at least one of the further objects which will become evident from the below description, is according to a first aspect of the technology proposed herein obtained by a modular CRISPR-Cas system comprising:
a CRISPR-Cas protein fused to a first binding peptide,
a functional protein fused to a second binding peptide,
   wherein the first binding peptide and the second binding peptide are configured to bind to each other and define a binding peptide pair.

The technology proposed herein is accordingly based on the recognition by the present inventors that the production of the fusion proteins of CRISPR-Cas and functional proteins is a limiting factor for using RNP delivery of a CRISPR-Cas system. Thus, it is understood that the modular CRISPR-Cas system is suitable for, or is arranged to be used in, ribonucleoprotein (RNP) delivery. Worded differently, the modular CRISPR-Cas system is an in vitro modular CRISPR-Cas system. Not only must the correct DNA sequences for the CRISPR-Cas protein and the functional protein be obtained and fused together, but the expression of the fused DNA sequence involves difficulties. If the desired peptide sequence corresponding to the fusion protein is obtained, then it is difficult to isolate and purify these rather large fusion proteins from the host cell. Understandably, a high degree of purity is required, which results in a low yield of fusion protein. Finally, even if a successful transfection has been obtained with one fusion protein, the production process (fusing DNA, expressing fused DNA, purifying fusion protein) must start anew if a different fusion protein is to be used. Thus, if a first fusion protein comprising a first CRISPR-Cas and a first functional protein is available for use, then a large effort is needed if it is desired to for example use the first CRISPR-Cas with a second, different, functional protein.

The CRISPR-Cas system according to the first aspect of the technology proposed herein addresses all these limitations. By using the binding peptides, the CRISPR-Cas protein can be manufactured, i.e. expressed and purified separately from the functional protein which greatly simplifies the production process and in particular the purification due to the smaller size of the proteins compared to the fusion protein. Further, by providing the CRISPR-Cas protein separately from the functional protein it is easy to change or modify the function of the CRISPR-Cas protein by pairing it with different functional proteins. The pairing may be done by simply mixing the CRISPR-Cas protein with the functional protein. This thus allows for further expanding the capabilities of the CRISPR-Cas system as it allows for adding further functionality, including functionality not yet used with CRISPR-Cas systems based on fusion proteins. This provides novel and enhanced genome editing possibilities.

The technology proposed herein thus provides a modular CRISPR-Cas system where different CRISPR-Cas proteins and functional proteins can be combined to form ribonucleoproteins that are then provided to the cell to be modified. The combination of the CRISPR-Cas protein and the functional protein is done outside the cell, e.g., in vitro and is thus easy to perform. The modularity of the system is evident in that once various CRISPR-Cas proteins and functional proteins have been manufactured, they may be combined as desired to form different ribonucleoproteins without the need to edit or change the vectors and sequences used to express the CRISPR-Cas proteins and functional proteins. Further, the gRNA, which guides the CRISPR-Cas system is also combined with the CRISPR-Cas protein in vitro, thus making it simple to choose and/or change the target gene to be modified.

Further, by combining the CRISPR-Cas protein and functional protein, and also the gRNA, in vitro, it is possible to determine the sequential order of assembly of the ribonucleoprotein. This may be important if the functional protein itself has an RNA interaction or affinity in order to prevent premature complexation between the gRNA and the functional protein. Assembling the CRISPR-Cas-complex with gRNA in vitro, instead of in vivo, further may prevent the gRNA from interacting with other cell components such as protein, RNA, and DNA, that are present in a cell, i.e., in vivo. Assembling the CRISPR-Cas-complex with gRNA in vitro further allows control over the molar ratio of the components.

In summary, the technology proposed herein therefore provides multiple advantages over e.g., WO2021165508 and US20200017869.

The modular CRISPR-Cas system is suitable for ribonucleoprotein (RNP) delivery or is arranged to be used in, ribonucleoprotein (RNP) delivery. Accordingly, the CRISPR-Cas protein and the functional protein are in a form suitable for forming a ribonucleoprotein (RNP) that in turn is in a form suitable to be delivered to a cell.

In the context of the technology proposed herein, the equivalent terms “for ribonucleoprotein (RNP) delivery” and “suitable for ribonucleoprotein (RNP) delivery” and “arranged to be used in ribonucleoprotein (RNP) delivery” encompasses that ribonucleoprotein (RNP) delivery can be used with the modular CRISPR-Cas system. In other words, the modular CRISPR-Cas system can be used with ribonucleoprotein (RNP) delivery.

In the context of the technology proposed herein the terms CRISPR-Cas system and CRISPR system are used interchangeably.

In the context of the technology proposed herein the term modular when applied to the CRISPR-Cas system means that the CRISPR-Cas system encompasses different possible modules, where for example, the CRISPR-Cas protein with its first binding peptide defines a first module, and the functional protein with its second binding peptide defines a second module. Generally, the system may comprise further modules such as a third module defined by a further functional protein also having the second binding peptide configured to bind to the first binding peptide. The Modular CRISPR-Cas system thus allows for assembling a CRISPR-Cas-complex by choosing a desired CRISPR-Cas protein with its first binding peptide and assembling it with a desired functional protein with its second binding peptide, thereby assembling any desired CRISPR-Cas-complex tailored for e.g. base editing, prime editing, or other type of editing as desired.

Specifically, the choosing and assembling of the CRISPR-Cas proteins and functional proteins is carried out on the proteins and not on genes encoding the proteins. This allows for ease of assembly, i.e., by contacting such as by mixing, and does not require modification or the genes encoding the CRISPR-Cas proteins and functional proteins.

The CRISPR-Cas system may for example comprise a first plurality of first modules corresponding to different CRISPR-Cas proteins having different activity and a second plurality of second modules corresponding to functional proteins have different function, to allow the user of the system to obtain any desired combination of CRISPR-Cas protein and functional protein.

The CRISPR-Cas system may exist in assembled form as the CRISPR-Cas-complex, i.e. with the first binding peptide bound to the second binding peptide, or in non-assembled form, i.e. where the first binding peptide and the second binding peptide are not bound to each other and the CRISPR-Cas protein and the functional protein are thus not associated as a CRISPR-Cas-complex.

The CRISPR-Cas protein may for example be a catalytically dead Cas9 (dCas9) or a Cas9 nickase (nCas9). Generally, the CRISPR-Cas protein may be any CRISPR-Cas-related protein, or part thereof, that is an RNA-guided DNA endonuclease enzyme such as CRISPR-Cas9, CRISPR-Cas12a or variants thereof. The CRISPR-Cas protein may have both, one, or none, of the nuclease domains functional.

The CRISPR-Cas protein is fused, preferably by being expressed sequentially with, the first binding peptide.

The first binding peptide and the second binding peptide typically each have a length of 10-170 amino acids. The first binding peptide and the second binding peptide are configured to bind to each other. The binding may be the result of attractive forces between the side chains of the amino acids making up the respective binding peptide.

In the context of the technology proposed herein the term peptide covers peptides, polypeptides, and proteins.

The functional protein is a protein that is capable of altering or affecting the outcome of applying the CRISPR-Cas protein to DNA. The functional protein may thus be configured to affect the functioning of the CRISPR-Cas protein. Alternatively the functional protein is configured to affect how the organism, whose genome or DNA is affected by the CRISPR-Cas protein, handles the effect, such as a break, caused by the action of the CRISPR-Cas protein. The functional protein may thus for example affect how a break of the strands of DNA is repaired. Improving directed repair such as homology directed repair or homologous recombination are thus additional features that could be provided by a functional protein.

The first and second binding peptides together define a binding peptide pair. Different binding peptide pairs are known in other technical fields. Binding peptide pairs may further be constructed synthetically, by designing sequences of the first and second binding peptides so as to bind to each other by the side chains. In particular the first and second binding peptides may be constructed so as to assume secondary structure, i.e. as alpha-helixes or beta sheets, suitable for binding.

The binding peptide pair preferably comprises a Cohesin/Dockerin system.

As shown in the example, the Cohesin/Dockerin system, i.e. in which one of the first and second binding peptide comprises or consists of Cohesin, and the other comprises of or consists of Dockerin, works well in the CRISPR-Cas system.

Generally, the binding peptide pair may comprise a first binding peptide, having at least 90% sequence identity, such as at least 95% or at least 99% sequence identity, to one of Cohesin and Dockerin, and a second binding peptide having at least 90% sequence identity, such as at least 95% or at least 99% sequence identity, to the other of Cohesin and Dockerin.

In other words, the binding peptide pair may comprise a first binding peptide, having at least 90% sequence identity, such as at least 95% or at least 99%, or 100% sequence identity to one of SEQ ID NO: 19 and SEQ ID NO 21 (see below) and a second binding peptide having at least 90% sequence identity, such as at least 95% or at least 99% or 100% sequence identity to the other of SEQ ID NO: 19 and SEQ ID NO: 21.

The amino acid sequence of Cohesin domain Cip7 is given by SEQ ID NO: 19:

MAVRIKVDTVNAKPGDTVRIPVRFSGIPSKGIANCDFVYSYDPNV
LEIIEIEPGELIVDPNPTKSFDTAVYPDRKMIVFLFAEDSGTGAY
AITEDGVFATIVAKVKSGAPNGLSVIKFVEVGGFANNDLVEQKTQ
FFDGGVNVG

The DNA sequence use to express Cohesin domain Cip7 in the Example further below is given by SEQ ID NO: 20 in which nucleotides 31-459 (underlined) encode Cohesin:

ACGCCAGATCTGCACCACCACCACCACCACGCGGTGCGCATTAAA
GTGGATACCGTGAACGCGAAACCGGGCGATACCGTGCGCATTCCG
GTGCGCTTTAGCGGCATTCCGAGCAAAGGCATTGCGAACTGCGAT
TTTGTGTATAGCTATGATCCGAACGTGCTGGAAATTATTGAAATT
GAACCGGGCGAACTGATTGTGGATCCGAACCCGACCAAAAGCTTT
GATACCGCGGTGTATCCGGATCGCAAAATGATTGTGTTTCTGTTT
GCGGAAGATAGCGGCACCGGCGCGTATGCGATTACCGAAGATGGC
GTGTTTGCGACCATTGTGGCGAAAGTGAAAAGCGGCGCGCCGAAC
GGCCTGAGCGTGATTAAATTTGTGGAAGTGGGCGGCTTTGCGAAC
AACGATCTGGTGGAACAGAAAACCCAGTTTTTTGATGGCGGCGTG
AACGTGGGCGGTGGTTCTGGTCATATGGGTACCACCCAG

The amino acid sequence of Dockerin domain Doc1 is given by SEQ ID NO: 21:

MPPVLLGDVNGDGTINSTDLTMLKRSVLRAITLTDDAKARADVDK
NGSINSTDVLLLSRYLLRVIDKFPV

The DNA sequence used to express Dockerin domain Doc1 in the Example further below is given by SEQ ID NO: 22 in which nucleotides 167-373 (underlined) encode Dockerin. In the sequence, the sequence for Dockerin is preceded by the c-terminus of Cas9 (first 155 nucleotides) and followed by the sequence for NLS (nucleotides 373-394):

TCTTGGAGCTCCCGCTGCTTTTAAATATTTTGATACAACAATTGA
TCGTAAACGATATACGTCTACAAAAGAAGTTTTAGATGCCACTCT
TATCCATCAATCCATCACTGGTCTTTATGAAACACGCATTGATTT
GAGTCAGCTAGGAGGTGACGGTGGTTCTGGTCCGCCGGTGCTGCT
GGGCGATGTGAACGGCGATGGCACCATTAACAGCACCGATCTGAC
CATGCTGAAACGCAGCGTGCTGCGCGCGATTACCCTGACCGATGA
TGCGAAAGCGCGCGCGGATGTGGATAAAAACGGCAGCATTAACAG
CACCGATGTGCTGCTGCTGAGCCGCTATCTGCTGCGCGTGATTGA
TAAATTTCCGGTGCCCAAGAAGAAGAGGAAGGTGCTCGAGCACCA
C

The peptide binding pair may comprise other peptide systems in which a first peptide is configured to bind a second peptide. Possible systems further include the FK506 binding protein 12 (FKBP)-FKBP rapamycin binding (FRB) domain system.

Preferably, the functional protein is selected among the group consisting of:
a) deaminases, preferably modified deaminases,
b) reverse transcriptases,
c) methylases, and
d) homologous recombination and homology directed repair enhancing proteins.

Expressed differently, the functional protein preferably is a deaminase, a reverse transcriptase, a methylase, or a homologous recombination protein or a homology directed repair enhancing protein.

Each of these types of functional proteins affect or alter the outcome of applying the CRISPR-Cas protein to DNA.

Deaminases are for example used in base editing (BE). Examples include cytidine deaminases which deaminates cytidine (C) resulting in uracil (U). This functional protein may be used together with a dead Cas9 (dCas9). Another example is adenosine deaminase, which may be used together with a nickase (nCas9).

In prime editing (PE) the functional protein may be a reverse transcriptase, which is used together with a nickase (nCas9).

Methylases add methyl groups to adenine or cytosine bases within a recognition sequence and thus protect these bases.

Homologous recombination and homology directed repair enhancing proteins act by suppressing NHEJ repair mechanisms, improve the accessibility of DNA for homologous recombination or guide a repair template to the CRISPR-Cas interacting site.

It is further contemplated within the context of the technology proposed herein that the functional protein may alternatively be a RAD protein, such as a RAD51 protein. RAD proteins, such as RAD51 protein, may bind to the DNA at the site of a break and encase the DNA in a protein sheath. By the use of a RAD protein as functional protein, the effect of the CRISPR-Cas protein can thus be further modified. A bacterial similar protein to RAD is the RecA protein.

The modular CRISPR-Cas system according to the first aspect of the technology proposed herein may further comprise:
a guide RNA sequence complex bound to the CRISPR-Cas protein.

The guide RNA sequence (gRNA) guides the CRISPR-Cas protein to the target DNA.

The guide RNA may be provided bound to the CRISPR-Cas protein, or may alternatively be provided separate from the CRISPR-Cas protein so that it can be assembled with the CRISPR-Cas protein by contacting the guide RNA and the CRISPR-Cas protein. The guide RNA may further be functionalized by also providing a template for genome editing by prime editing, pegRNA.

Preferably, the modular CRISPR-Cas system according to the first aspect of the technology proposed herein comprises:
a first container containing the CRISPR-Cas protein fused to the first binding peptide,
a second container containing the functional protein fused to the second binding peptide,

This is advantageous in that it allows for users of the modular CRISPR-Cas system combine CRISPR-Cas protein with the desired functional protein. An embodiment of the modular CRISPR-Cas system, such as a kit, may thus be provided which allows the user to perform both base editing and prime editing, by selecting the corresponding CRISPR-Cas protein and functional protein, from the same kit. The system may thus be implemented as a kit comprising at least one CRISPR-Cas protein with its corresponding first binding peptide, more preferably at least two such as one dCas9 and one nCas9, and at least one functional protein with its corresponding second binding peptide, preferably at least two such as one deaminase and one reverse transcriptase.

The first and second container may be any type of container, but preferably a test tube such as a plastic test tube, for example an Eppendorf tube, or a glass test tube, that is capable of holding the CRISPR-Cas protein or the functional protein. The CRISPR-Cas protein and the functional protein may thus be provided in a buffer or solution. It is further contemplated that the CRISPR-Cas protein and the functional protein may be provided in solid form, such as dried form or crystal form.

Preferably the modular CRISPR-Cas system according to the first aspect of the technology proposed herein comprises:
a further functional protein fused to a third binding peptide, wherein the further functional protein is different from the functional protein, and wherein the third binding peptide is configured to bind to the first binding peptide, and wherein the third binding peptide preferably has the same amino acid sequence as the second binding peptide, and wherein the modular CRISPR-Cas system preferably further comprises:
a third container containing the further functional protein fused to the third binding peptide.

This is advantageous in that it provides the basic option to choose which functional protein to use with the CRISPR-Cas protein. The third container may be of the same type as the second container described above.

The third binding peptide is configured to bind to the first binding peptide. The third binding peptide may have an amino acid sequence that has a sequence identity of at least 90%, such as at least 95%, more preferably at least 99%, to the amino acid sequence of the second binding peptide. Even more preferably the third binding peptide has the same amino acid sequence as the second binding peptide.

It is further possible that the system comprises two or more sets of binding peptide pairs. In that case each set of binding peptide pairs comprises a first binding peptide fused to the CRISPR-Cas protein and a second binding protein fused to a functional protein as described above. This allows the use of multiple functional proteins with the CRISPR-Cas protein. In this case it is preferred that the sequences of the first and second binding peptides of each set of binding peptide pairs are configured so that the first binding peptide of a first set of binding peptide pairs only binds to the second binding peptide of the same set of binding peptide pairs, and accordingly does not bind to the second binding peptide of another set of binding peptide pairs.

Preferably the further functional protein fused to the third binding peptide is provided in its own buffer or provided in solid form, such as dried form or crystal form.

The second aspect of the technology proposed herein concerns a method of assembling a CRISPR-Cas-complex comprising the steps of:
i. providing the system according to the first aspect of the technology proposed herein, and
ii. contacting the CRISPR Cas protein fused to the first binding peptide, and the functional protein fused to the second binding peptide, with each other,
   wherein the method preferably further comprises the step of:
iii. contacting the CRISPR Cas protein with a guide RNA sequence.

Step iii may be performed prior to step ii.

Thus, it is understood that the CRISPR-Cas complex is suitable for, or is arranged to be used in, ribonucleoprotein (RNP) delivery. Worded differently, the CRISPR-Cas complex is an in vitro modular CRISPR-Cas system.

The contacting of the CRISPR-Cas protein with its first binding peptide and the functional protein with its second binding peptide is preferably performed by mixing. The CRISPR-Cas protein with its first binding peptide and the functional protein with its second binding peptide are thus preferably provided in separate buffer solutions, such that the buffer solutions can be mixed to assemble the CRISPR-Cas-complex.

Alternatively, the CRISPR-Cas protein with its first binding peptide and the functional protein with its second binding peptide may be provided in crystal form as solids, and mixed together in a buffer to assemble the CRISPR-Cas-complex.

Similarly, the guide RNA sequence may be provided in a buffer for mixing with the CRISPR-Cas protein. As described above, the guide RNA may be modified, e.g. as pegRNA.

Step ii of contacting the CRISPR-Cas protein with its first binding peptide and the functional protein with its second binding peptide, is performed in vitro. In other words, the contacting is performed outside the cell to which the CRISPR-Cas-complex is to be delivered as a ribonucleoprotein.

Step iii of contacting the CRISPR Cas protein with the guide RNA sequence may be performed prior to, or after step ii of contacting the CRISPR Cas protein fused to the first binding peptide, and the functional protein fused to the second binding peptide, with each other. Step iii is performed in vitro. In other words, the contacting is performed outside the cell to which the CRISPR-Cas-complex is to be delivered as a ribonucleoprotein.

Performing step iii before step ii may be advantageous when the functional protein itself has an RNA interaction or affinity in order to prevent complexation between the gRNA and the functional protein. Assembling the CRISPR-Cas-complex with gRNA in vitro, instead of in vivo, further may prevent the gRNA from interacting with other cell components such as protein, RNA, and DNA, that are present in a cell, i.e., in vivo.

The third aspect of the technology proposed herein concerns a CRISPR-Cas complex obtained by the method according to the second aspect of the technology proposed herein, i.e. a CRISPR-Cas complex for ribonucleoprotein (RNP) delivery comprising:
a CRISPR-Cas protein fused to a first binding peptide,
a functional protein fused to a second binding peptide,
   wherein the first binding peptide and the second binding peptide are bound to each other, and wherein the CRISPR-Cas complex preferably further comprises:
a guide RNA sequence complex bound to the CRISPR-Cas protein.

Thus it is understood that the CRISPR-Cas complex is suitable for, or is arranged to be used in, ribonucleoprotein (RNP) delivery. Worded differently, the CRISPR-Cas complex is an in vitro modular CRISPR-Cas system.

The CRISPR-Cas complex corresponds to the system according to the first aspect of the technology proposed herein when the first and the second binding peptides are bound to each other. The CRISPR-Cas complex can be used to modify DNA in vivo or in vitro, and the DNA can be free (isolated DNA) or in a cell or an organism (genomic DNA). The fourth aspect of the technology proposed herein concerns the use of the modular CRISPR-Cas system according to the first aspect of the technology proposed herein, or the CRISPR-Cas complex according to the third aspect of the technology proposed herein, for modifying the genome of an organism, wherein the organism preferably is a plant selected from the group consisting of a monocot and dicot plant, or the organism is a plant selected from the group consisting of maize, rice, sorghum, rye, barley, wheat, millet, oats, sugarcane, turfgrass, or switchgrass, soybean, canola, alfalfa, sunflower, cotton, tobacco, peanut, potato, sugar beet, grape, Arabidopsis and safflower cell, and wherein preferably the organism is a plant from the family Solanaceae, preferably from the genus Solanum, more preferably from the species Solanum tuberosum.

The use may comprise using the modular CRISPR-Cas system according to the first aspect of the technology proposed herein, or the CRISPR-Cas complex according to the third aspect of the technology proposed herein, for modifying the genome of a cell of the organism, such as a plant cell. Once a plant having had its genome modified has been obtained, then the plant cell may be cultivated as known in the art to obtain a plant. The finished plant can then in turn be propagated and/or cultivated to obtain further plants for planting and farming.

The organism is preferably not a human embryo. The use according to the fourth aspect of the technology proposed herein preferably does not comprise modifying the germ-line genetic identity of humans. Further, the use according to the fourth aspect of the technology proposed herein preferably does not comprise a surgical or therapeutic method for the treatment of the human or animal body.

A fifth aspect of the technology proposed herein thus relates to a plant obtained by the use according to the fourth aspect of the technology proposed herein.

Correspondingly, a sixth aspect of the technology proposed herein concerns a plant comprising or consisting of one or more plant cells having had its genome modified by the use according to the fourth aspect of the technology proposed herein.

A seventh aspect of the technology proposed herein concerns progeny of a plant according to the fifth or sixth aspect of the technology proposed herein.

An eight aspect of the technology proposed herein concerns a seed or spore obtained from a plant according to the fifth or sixth aspect of the technology proposed herein.

A ninth aspect of the technology proposed herein concerns a vegetatively propagation-capable object, such as a cutting or graft or tuber or bulb or callus, of a plant according to the fifth or sixth aspect of the technology proposed herein.

### BRIEF DESCRIPTION OF THE DRAWINGS AND DETAILED DESCRIPTION

A more complete understanding of the abovementioned and other features and advantages of the technology proposed herein will be apparent from the following detailed description in conjunction with the appended drawings, wherein:

FIG. 1A shows a Cas9 protein assembled with a green fluorescent protein (GFP) by first and second binding peptides shown as a Dockerin peptide (D) and a cohesion peptide (C).

FIG. 1B shows a Cas9 complex comprising a Cas9 protein assembled with a reverse transcriptase (RT) by first and second binding peptides shown as a Dockerin peptide (D) and a cohesion peptide (C).

FIG. 2A shows a schematic overview of the three cloned constructs. Top: Cas9-Doc1 (Cas 9 with 2.1 kbp corresponding to 158 kDa, Doc 1 with 0.2 kbp corresponding to 7 kDa), Middle: Cip7-GFP (Cip7 with 0.4 kbp corresponding to 15 kDa, GFP(GFPuv) with 0.7 kDa corresponding to 27 kDa) Bottom: Cip7-RT (Cip7 with 0.4 kbp corresponding to 15 kDa, RT (MMLV-RT) with 2.1 kbp corresponding to 77 kDa). In each of the constructs the T7 promoter controls the expression, allowing for inducible protein expression in Rosetta (DE3) E. coli strain. Kanamycin resistance (KanR) is used as a selection marker in all constructs. A nuclear localization sequence (NLS) is followed by the SpCas9 gene, which is fused to the first adaptor component Doc1. The second adaptor component Cip7 is fused to either GFP or Moloney Murine Leukemia Virus Reverse Transcriptase (MMLV-RT).

FIG. 2B shows a restriction analysis to verify the structure of the plasmid and successful transformation. Plasmid DNA was purified from transformed and untransformed (untransf.) Rosetta (DE3) cells, digested with HindIII and BglII and restriction patterns compared to the respective plasmid used for transformation. The band likely representing pRARE is indicated with an arrow.

FIG. 3 shows a Cas9 in-vitro cleavage assay to confirm nuclease activity. a Patatin 1 PCR fragment was incubated either with commercial TrueCut Cas9 or purified Cas9-Doc1 and gRNA. The reactions were run on an agarose gel. b Sequence of the gRNA and position in the target DNA fragment.

FIG. 4 shows a PAGE showing interaction of Cip7-GFP and Cas9-Doc1. Cip7-GFP and Cas9-Doc1 were loaded to a native gel, with increasing amounts of the latter one. TrueCut Cas9 was used as a negative control. Depicted values are in pmol. After electrophoresis the gel was imaged for GFP signal.

FIG. 5 shows a Cas9 electrophoretic mobility shift assay (EMSA) with imaging for GFP. The Cas9 in-vitro cleavage assay (FIG. 3) was adapted as an EMSA by removing the final heating step, which allows Cas9 to stay associated with the target DNA causing a mobility shift. After electrophoresis the gel was imaged for GFP signal and subsequently DNA was stained using a GelRed post-staining protocol. The GFP band of the apparent interaction between Cip7-GFP+Cas9-Doc1 is indicated with an arrow.

FIG. 6 shows Cas9 electrophoretic mobility shift assay (EMSA) with Cip7-GFP and Cip7-RT. The experimental setup is similar to FIG. 5 but less DNA and protein was used, and the running time was extended to achieve better separation. The amount of Cas9-Doc1 was kept the same while increasing Cip7-GFP or Cip7-RT. TrueCut Cas9 was used as a control. Cas9-Doc1 or TrueCut Cas9 associated with the target DNA is indicated by the lower arrows in the respective panel, and the complexes with Cip7-RT or Cip7-GFP are indicated by the upper arrows in the respective panel.

FIG. 7A shows RNP transfected potato protoplasts in different conditions in bright field (left) and UV light (right). a, negative control (NC) displays no GFP signal. b, in Cip7-GFP transfected protoplasts GFP signal is clearly visible. c, protoplasts transfected with Cip7-GFP and Cas9-Doc1 showed more frequent green fluorescence in comparison to just Cip7-GFP. Scale bar 150 μm.

FIG. 7B shows potato protoplasts transfected with both Cip7-GFP and Cas9-Doc1 in bright field (left) and in UV light (right). a, intact protoplasts with strong autofluorescence did not give any apparent sign of GFP signal. b, protoplasts with less autofluorescence appeared more yellow to green in color which indicated the presence of GFP. c, broken protoplasts and cell debris showed increased green fluorescence. Scale bar 10 μm.

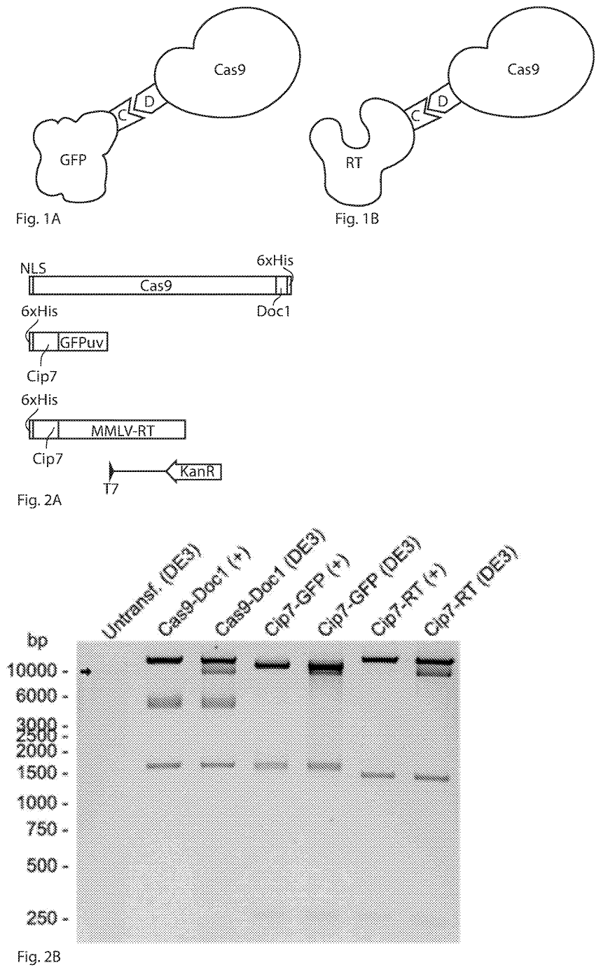

### EXAMPLE—CREATING A MODULAR CRISPR SYSTEM

### 1. Materials and Methods

### 1.1 Oligonucleotide Annealing for Cloning

Oligonucleotides were diluted to 100 μM and 20 μL of both strands mixed with 20 μL 5M NaCl solution and 20 μL water (Sequences are listed in Table 1 below). The mix was heated to 95 °C and cooled down to room temperature. The annealed DNA fragments were diluted to suitable concentrations and used for cloning.

### 1.2 Molecular Cloning

TABLE 1
The fragments and plasmids that were used for cloning

| Name | Description | Origin |
|---|---|---|
| pMJ806 T7 | expression vector carrying the SpCas9 gene | Addgene #39312 |
| pET30-R5- | T7 expression vector carrying the GFP gene | Addgene #17838 |
| GFP | | |
| RBS- | Forward oligonucleotide carrying RBS and NLS: | Oligonucleotide |
| NLS_F | CTAGATTTGTTTAACTTTAAGAAGGAGATATACCA | annealing |
| | TGCCCAAGAAGAAGAGGAAGGTGACTAGTCCA | |
| | (SEQ ID NO: 1) | |
| RBS- | Reverse oligonucleotide carrying RBS and NLS: | Oligonucleotide |
| NLS_R | GATCTGGACTAGTCACCTTCCTCTTCTTCTTGGGC | annealing |
| | ATGGTATATCTCCTTCTTAAAGTTAAACAAAT | |
| | (SEQ ID NO: 2) | |
| RBS_F | Forward oligonucleotide carrying RBS: | Oligonucleotide |
| | CTAGATTTGTTTAACTTTAAGAAGGAGATATACCA | annealing |
| | TGACTAGTCCA | |
| | (SEQ ID NO: 3) | |
| RBS_R | Reverse oligonucleotide carrying RBS: | Oligonucleotide |
| | GATCTGGACTAGTCATGGTATATCTCCTTCTTAAA | annealing |
| | GTTAAACAAAT | |
| | (SEQ ID NO: 4) | |
| Doc1 | DNA fragment carrying the Doc1 gene | IDT gBlock |
| Cip7 | DNA fragment carrying the Cip7 gene | IDT gBlock |

pMJ806 (Addgene #39312), a T7 expression vector carrying the SpCas9 gene, was digested using ThermoFisher FastDigest restriction enzymes (RE) XhoI and SpeI. The fragments were separated by 1.2% agarose gel electrophoresis and purified using the ThermoFisher GeneJET Gel Extraction Kit according to the manufacturer's instructions. DNA concentration was determined by spectrophotometry using a DeNovix DS-11 device.

The 83 bp DNA fragment RBS-NLS (Table 1) created by oligonucleotide annealing was digested using XhoI and SpeI and purified with the ThermoFisher GeneJet PCR Purification kit. Ligation was performed using the ThermoFisher T4 DNA ligase in a 20 μL reaction with a plasmid:insert ratio of 1:50. The ligation reaction was diluted 5 times and 2 μL were used to transform ThermoFisher OneShot Top10 chemically competent E. coli cells according to the manufacturer's instructions by 42 °C heat-shock. 75 μL of transformed cells were plated on LB agar plates supplemented with 50 μg/mL kanamycin and incubated overnight at 37 °C. Colonies were picked to inoculate 5 mL LB overnight cultures, plasmids were purified using the ThermoFisher GeneJET plasmid miniprep kit and correct ligation was verified by restriction analysis.

The IDT gBlock Doc1-NLS DNA fragment (Table 1) was inserted using SacI and XhoI by digestion and ligation as mentioned previously. For ligation a plasmid:insert ratio of 1:3 was used, creating the pMC:RBS-NLS-Cas9-Doc1 construct. pMC:Cip7-GFP was created by removing undesired tags in pET30-R5-GFP (Addgene #17838) and replacing them with the 46 bp DNA fragment RBS (oligonucleotide annealing) using XbaI and BglII with a plasmid:insert ratio of 1:50 as described earlier. In a second step, Cip7 from a synthesized DNA fragment (IDT gBlock) was introduced using BglII and KpnI as described earlier with a plasmid:insert ratio of 1:3. The MMLV-RT from pET-19b_MMLV-RT (Addgene #153312) was cut out using NdeI and XhoI and ligated into pMC:Cip7-GFP with plasmid:insert ratio of 1:3, replacing the GFP gene and creating pMC:Cip7-RT. Plasmid sequences were confirmed by Sanger sequencing with primers listed in Table 2.

TABLE 2
Primers used for sequencing

| Name | Sequence | SEQ ID NO: |
|---|---|---|
| RBS-NLS-Cas9-Doc1 #1 | GGCGTAGAGGATCGAGAT | SEQ ID NO: 5 |
| RBS-NLS-Cas9-Doc1 #2 | CAAGTGGAGTAGATGCTA | SEQ ID NO: 6 |
| RBS-NLS-Cas9-Doc1 #3 | TGCATGGATGACTCGGAA | SEQ ID NO: 7 |
| RBS-NLS-Cas9-Doc1 #4 | GACAAGGCGATAGTTTACA | SEQ ID NO: 8 |
| RBS-NLS-Cas9-Doc1 #5 | AAACTCGCCAAATCACTAAG | SEQ ID NO: 9 |
| RBS-NLS-Cas9-Doc1 #6 | CGTTAAAGAGTTACTAGGGA | SEQ ID NO: 10 |
| RBS-Cip7-GFP #1 = | GGCGTAGAGGATCGAGAT | SEQ ID NO: 11 |
| RBS-Cip7-RT #1 | | |
| RBS-Cip7-GFP #2 | CACTTGTCACTACTTTCTCT | SEQ ID NO: 12 |
| RBS-Cip7-RT #2 | ACGAAACCAGCAAAGAACC | SEQ ID NO: 13 |
| RBS-Cip7-RT #3 | TTTCAAAAACTCACCTACCC | SEQ ID NO: 14 |
| RBS-Cip7-RT #4 | GGTAGAAGCCCTGGTGAAA | SEQ ID NO: 15 |

### 1.3 Preparation and Transformation of MilliporeSigma Novagen Rosetta (DE3) Competent Cells

Freezer stocks of MilliporeSigma Novagen Rosetta (DE3) E. coli cells were streaked on LB agar plates and incubated overnight at 37 °C. Colonies were used to inoculate 5 mL LB cultures, that were incubated at 37 °C and 225 rpm overnight. The next day 50 mL LB medium was inoculated with 0.5 mL overnight culture and incubated for 2 h at 37 °C and 225 rpm.

The following steps were performed at 4 °C. Cultures were chilled on ice for 10 min, centrifuged for 10 min at 4000×g and the supernatant decanted. The pellet was resuspended in 10 mL icecold TFB1 buffer (30 nM KOAc, 100 mM RbCl, 10 mM CaCl₂ 2H₂O, 50 mM MnCl₂·4H₂O, 15% glycerol) and incubated on ice for 5 min. Cells were centrifuged for 10 min at 4000×g, the supernatant decanted and the pellet was resuspended in 2 mL TFB2 (10 mM MOPS, 75 mM CaCl₂·2H₂O, 10 mM RbCl, 15% glycerol), with subsequent incubation on ice for 15 min. The cells were aliquoted, flash-frozen in liquid nitrogen and stored at −80 °C. 10 ng of purified plasmid was added to 50 μL competent cells and chilled on ice for 30 min, subsequently incubated for 30 s at 42 °C and put quickly back on ice. 100 μL ThermoFisher S.O.C. medium was added and cells were grown for 1 h at 37 °C and 225 rpm. 75 μL of cells were plated on LB agar plates supplemented with 50 μg/mL kanamycin and incubated overnight at 37 °C.

### 1.4 Protein Expression and Purification

Colonies from LB agar plates were picked and used for inoculation of 5 mL TB medium (Terrific Broth: 1.2% tryptone, 2.4% yeast extract, 0.5% glycerol, 89 mM), supplemented with 50 μg/mL kanamycin and incubated overnight at 37 °C and 225 rpm. 50 mL TB medium, supplemented with 50 μg/mL kanamycin, was inoculated with 0.5 mL overnight culture and grown at 37 °C until OD600=0.6 was reached. Protein expression was induced with 0.1 mM IPTG, the temperature reduced to 18 °C and cultures were grown for 20 h at 225 rpm. Cells were harvested by centrifugation at 4000×g for 20 min, the supernatant decanted and the pellet flash-frozen in liquid nitrogen. The pellet was resuspended in 5 mL binding buffer (Cytiva His phosphate buffer, pH 7.4, 80 mM imidazole, 1× Thermo Scientific™ Halt™ Protease Inhibitor Cocktail). Resuspended cells were lysed by sonication using the Hielscher UP400St ultrasonicator with 400 Ws/mL. The lysate was clarified by centrifugation for 15 min at 11.000×g. The clarified lysate was applied to Cytiva His Spintrap columns and purification performed according to the manufacturer's instructions. Briefly, the storage solution was removed and the column equilibrated with binding buffer. The clarified lysate was applied and the column washed once with binding buffer. Bound protein was eluted using elution buffer (Cytiva His phosphate buffer, pH 7.4, 500 mM imidazole) in two fractions.

Protein concentration was determined using the Thermo Scientific Coomassie (Bradford) protein assay kit according to the manufacturer's instructions. The standard microplate protocol with a working range of 100-1500 μg/mL was used and the absorbance measured at 595 nm in a Thermo Scientific Multiskan GO microplate spectrophotometer. The purification process was evaluated utilizing sodium dodecylsulfate polyacrylamide gel electrophoresis (SDS-PAGE).

### 1.5 SDS-PAGE

Protein samples were mixed with Novex™ Tris-Glycine SDS Sample Buffer (2×), incubated at 85 °C for 5 min and loaded to a 10% Novex™ Tris-Glycine gel. Unstained Thermo Scientific PageRuler was loaded to determine the molecular weight. The gel was run in Novex™ Tris-Glycine SDS Running Buffer at 120 V until the dye front reached the bottom of the gel. The gel was carefully removed from the plastic case, washed thrice in MilliQ water for 5 min and stained for 1 h in GelCode™ Blue Stain Reagent. Imaging was performed using a Bio-Rad ChemiDoc Touch Imaging System. Running distances were measured in Fiji ImageJ, a standard curve was created in Microsoft Excel by linear regression and molecular weights of the purified protein estimated:

MW=10^{(mx+n)} (1)

where m is the slope, x the running distance of the band of interest and n the intercept of the standard curve. The standard error on slope and intercept were propagated to calculate the error on the estimated molecular weights following the rules of gaussian error propagation:

$$Δ MW = \sqrt{{( \frac{\partial MW}{\partial m} Δ m )}^{2} + {( \frac{\partial MW}{\partial n} Δ n )}^{2} + {( \frac{\partial MW}{\partial x} Δ x )}^{2}} \qquad ( 2 )$$

### 1.6 Cas9 In-Vitro Cleavage Assay

The protocol was adapted from New England Biolabs, 2014. As DNA substrate the patatin-1 gene was amplified by PCR (7 s 98 °C, 20 s 60 °C, 25 s 72 °C, 30 cycles) using PatF: ATGGCAACTACTAAATCTTTTTTAAT (SEQ ID NO: 16) and PatR: TTAATAAGAAGCTTTGTTTGCTCGG (SEQ ID NO: 17) from an in-house unpublished plasmid and purified using the ThermoFisher GeneJET PCR cleanup kit. The previously validated gRNA (PatActT1: GTAATTGGAGGAACAAGTAC (SEQ ID NO: 18)) was used to confirm the activity of the Cas9 nuclease.

To assemble the RNP, 30 nM Cas9 (ThermoFisher TrueCut Cas9 v2 as control or purified Cas9-Doc1) and 30 nM gRNA were mixed and incubated at 25 °C for 10 min in Cas9 nuclease reaction buffer (20 mM HEPES, 100 mM NaCl, 5 mM MgCl2, 100 nM EDTA). Subsequently, substrate DNA was added in a final concentration of 3 nM giving a ratio of 10:10:1 (Cas9:gRNA:substrate DNA) and the reaction incubated for 1 h at 37 °C. Then 1 μL proteinase K was added and incubated at 65 °C for 10 min and finally the reaction was analyzed on a 2% agarose gel.

### 1.7 Native PAGE

Protein samples were diluted to 0.5-2 pmol/μL concentrations, mixed with sample buffer (final concentration: 100 mM Tris HCl, 10% glycerol, 0.000 25% bromophenol blue, pH 8.6) and incubated for 5 min at RT. Samples were loaded onto 10% Invitrogen Novex Tris-glycine gels and run in native running buffer (25 mM Tris base, 192 mM glycine, pH 8.3) at 200 V on ice until the dye front reached the bottom of the gel. Imaging for GFP was performed using a Bio-Rad ChemiDoc Touch Imaging System in the Pro-Q Emerald 488 settings of the device.

### 1.8 Cas9-Electrophoretic Mobility Shift Assay (EMSA)

The Cas9 in-vitro cleavage assay was adapted to investigate interaction of Cas9-Doc1 with Cip7-GFP and Cip7-RT. To visualize for GFP, 2 pmol of both Cas9-Doc1 and Cip7-GFP per 20 μL reaction was used in order to achieve a prominent GFP signal. For more distinct bands in Biotium GelRed imaging, 1 pmol of Cas9-Doc1 and increasing concentrations of Cip7-GFP or Cip7-RT (1, 2, 4, 6, 8 μmol) were used. ThermoFisher TrueCut v2 Cas9 was used as a control. Cas9-Doc1, Cip7-GFP or Cip7-RT and gRNA were mixed and pre-incubated for 10 min at 25 °C in Cas9 reaction buffer. Target DNA was added in a molar ratio 10:10:1 (Cas9:gRNA:DNA) after pre-incubation and the reaction incubated at 37 °C for exactly 30 min. The tubes were immediately chilled on ice before loading on an 1% agarose gel, which was run at 4 V/cm. The GFP signal was imaged using a Bio-Rad ChemiDoc Touch Imaging System in the Pro-Q Emerald 488 settings of the device. Post-staining was performed in 3× Biotium GelRed solution for 20 min after which the gel was imaged using the Biotium GelRed settings of the device.

### 1.9 Protoplast Isolation, Transfection and Regeneration

Protoplasts were isolated, transfected and regenerated according to the protocol by Andersson et al., 2018. Briefly, leaves from 4 to 6 weeks old in-vitro S. tuberosum cv. Desiree plants were used for protoplast isolation. 1-2 g of leaves were incubated for 24 h at 4 °C in a petri dish with 20 mL conditioning medium (2.7 g/L MS salts, 0.001% Nitsch & Nitsch vits, 100 mg/L casein hydrolysate, 2 mg/L NAA, 0.5 mg/L BAP, pH 5.8). Leaves were cut into 1 mm wide slices and incubated for 30 min in 20 mL plasmolysis solution (0.5M D-sorbitol). The solution was replaced with 25 mL enzyme solution (1× Macro, 1×Fe/EDTA, 1× Micro, 0.5×Vits 1, 0.5×Vits 2, 0.5×Vits 3, 5×Nitsch & Nitsch vits, 1× Organics, 500 mg/L Casein hydrolysate, 0.205M D-glucose-monohydrate, 0.205M Mannitol, 20 g/L PVP-10, 5.37 μM 1-Naphthaleneacetic acid, 1.78 μM 6-Benzylaminopurine, 10 g/L Cellulase R10, 2 g/L Maceroen-zyme R10, 6 mM CaCl2·2H2O, pH 5.6) and incubated for 14 h at 25 °C. Protoplast solution was filtered through 100 μm and 70 μm filters before centrifuging for 5 min at 50×g. The protoplast pellet was resuspended in 2 mL of wash solution (1× Macro, 6 mM CaCl2·2H2O, 1× Micro, 240 mM NaCl, 10.74 μM 1-Naphthaleneacetic acid, 2.22 μM 6-Benzylaminopurine, 1×Fe/EDTA, pH 5.6) and carefully applied to tubes containing 6 mL sucrose solution (0.43M Sucrose). Centrifugation was done for 15 min at 50×g. Protoplasts were carefully moved to transformation buffer 1 (190 mM Mannitol, 100 mM Cacl2·2H2O, 0.5% MES, pH 5.6) and counted in a Fuchs-Rosenthal counting chamber, centrifuged for 10 min at 50×g and resuspended in transformation buffer 2 (0.5M Mannitol, 15 mM MgCl2·6H2O, 0.1% MES, pH 5.6) to a concentration of 1.6·106 pps/mL. 5 μg of Cas9 was pre-incubated for 15 min at room temperature (RT) with 1.5 nmol gRNA (PatActT1: GTAATTGGAGGAACAAGTAC (SEQ ID NO: 18)) and 10 μg Cip7-GFP or reaction buffer (20 mM HEPES, 100 mM NaCl, 5 mM MgCl2, 100 nM EDTA) as control. 100 000 pps were added to each reaction together with 120 μL 40% polyethylene glycol (PEG) and incubated for 30 min at RT. 5 mL wash solution was added and protoplasts were centrifuged for 5 min at 50×g. For microscopy protoplasts were resuspended in 50 μL of wash solution and imaged using a Leica DMLB fluorescence microscope. For cell division, the protoplasts were resuspended in 1 mL culture medium E (1× Macro, 1×Fe/EDTA, 2.5 mM CaCl2·H2O, 1× Micro, 0.5×Vits 1, 0.5×Vits 2, 0.5×Vits 3, 1× Sugars, 1× Organics, 500 mg/L Casein hydrolysate, 0.17M D-Glucose monohydrate, 0.17M Mannitol, 2 g/L Bovine serum albumin, 5.37 μM 1-Naphthaleneacetic acid, 1.78 μM 6-Benzylaminopurine, pH 5.6) and 1 mL alginate solution (2.8% alginic acid sodium salt, 0.4M Sorbitol) was added for embedding. The protoplast were incubated in the dark at 25 °C for 5 days. In the following 14 days the light was stepwise increased to reach 10 μmol/m2/s. The emerging calli were imaged and counted using the Analyze Particles tool in Fiji ImageJ.

### 1.10 Statistical Analysis

One-way ANOVA was performed in RStudio (R Core Team, 2022) with subsequent pairwise T-test comparison using the Bonferroni correction method.

### 1.11 Solutions Used

Solutions used and referred to above are defined in Table 3 below:

TABLE 3
Solutions used

| Solution | Recipe for 100 mL |
|---|---|
| 100x Macro | 7400 mg KNO3, 4920 mg MgSO4, 340 mg |
| | KH2PO4 |
| 100x FE/EDTA | 140 mg, Na2EDTA, 190 mg FeSO4 |
| 1000x Micro | 150 mg H3BO3, 500 mg MnSO4, 100 mg |
| | ZnSO4, 12 mg |
| | Na2MoO4, 1.2 mg CuSO4, 1.2 mg |
| | CoCL2, 38 mg KI |
| 1000x Nitsch | 100 mg Glycine, 5000 mg Myo-Inositol, 25 |
| & Nitsch vits | mg Thiamin-HCL, |
| | 25 mg Pyridox-HCL, 250 mg Nicotinic |
| | acid, 25 mg Folic acid, |
| | 2.5 mg Biotin |
| 100x Vits 1 | 50 mg Pantothenoic acid, 50 mg Choline |
| | chloride, 100 mg |
| | Asorbic acid, 1 mg p-Aminobenzoic acid, |
| | 50 mg Nicotinic acid, |
| | 50 mg Pyridoxine, 500 mg Thiamine |
| 100x Vits 2 | 1 mg p-Aminobenzoic acid, 50 mg |
| | Nicotinic acid, 50 mg Pyridoxine, |
| | 500 mg Thiamine |
| 100x Vits 3 | 0.5 mg Vitamin D3 |
| 50x Sugars | 625 mg Sorbitol, 625 mg Sucrose, 625 mg |
| | Fructose, 625 mg |
| | Ribose, 625 mg Xylose, 625 mg Mannose, |
| | 625 mg Rhamnose, |
| | 625 mg Cellobiose, 250 mg Myo-Inositol |
| 100x Organics | 100 mg Pyruvic acid, 200 mg Fumaric |
| | acid, 200 mg Citric acid, |
| | 200 mg Malic acid |

### 2. Results

### 2.1 Expression Constructs

Three expression constructs were created using molecular cloning as a first step towards a modularly interactive CRISPR system for RNP delivery (FIG. 2A). In a T7 expression vector carrying the gene for SpCas9, undesired tags in the N-terminus were replaced with a short sequence including a Ribosomal Binding Site (RBS) and a Nuclear Localization Sequence (NLS). To the C-terminus the dockerin domain Doc1 was added creating the first construct Cas9-Doc1 (FIG. 2A, top). To a second T7 expression vector carrying the gene for GFPuv, again an RBS was added to the N-terminus along with the cohesin domain Cip7 which gave the second construct Cip7-GFP (FIG. 2A, middle). In a last step the GFPuv was replaced with the Moloney Murine Leukemia Virus Reverse Transcriptase (MMLV-RT) creating Cip7-RT (FIG. 2A, bottom).

Using the T7 expression system these constructs were used to produce recombinant proteins in E. coli and subsequent RNP delivery in potato protoplasts. All three constructs are under the control of the T7 promoter that is recognized by the T7 RNA polymerase and allows for tightly controlled gene expression in E. coli strains carrying the gene for the T7 RNA polymerase. Furthermore, all three constructs have kanamycin resistance (KanR) for selection of successfully transformed colonies. The first construct begins with a NLS to signal import of the recombinant protein into the cell nucleus. The SpCas9 gene is adjacent to the NLS and codes for the SpCas9 protein, which is the core of the CRISPR system. The dockerin domain Doc1 represents a first binding peptide, also called adapter herein, and will enable to couple other functional proteins to Cas9. Lastly, the 6×His-tag allows for protein purification. The recombinant protein produced by this construct will hereafter be referred to as Cas9-Doc1. The second construct starts with the 6×His-tag for purification, which is followed by the second binding peptide or second adapter component, the cohesin domain Cip7 that can bind to the Doc1 domain. GFPuv is a variant of the wild-type green fluorescent protein (GFP) and optimized for excitation by UV light. Here, it acts as a reporter in a proof-of-principle phase. The respective recombinant protein will be named Cip7-GFP. The last construct is similar to the second, but GFP is replaced with the MMLV-reverse transcriptase, that is used in prime editing systems. Hereafter, it will be called Cip7-RT.

Rosetta (DE3) E. coli cells were transformed with the constructed expression vectors and successful transformation was verified by restriction analysis (FIG. 2B). Plasmid DNA was isolated from transformed and untransformed Rosetta (DE3) cells. Isolated plasmid DNA was digested using HindIII and BglII together with the respective plasmid used for transformation as positive control. The restriction patterns of isolated DNA from Rosetta (DE3) cells were for all three constructs almost identical to the control. However, an additional band at about 10 000 bp could be observed in all samples isolated from Rosetta (DE3) cells including the untransformed control (FIG. 2B, indicated with an arrow). This band can most likely be attributed to the pRARE plasmid that the Rosetta (DE3) strain carries for expression of rare tRNAs. The results from the restriction analysis indicated successful transformation and allowed to start protein expression.

### 2.2 Protein Expression and Purification

Overnight cultures of transformed bacteria were used to inoculate bacterial culture medium. Cultures were grown until OD600=0.6 was reached, protein expression was induced using 0.1 mM Isopropyl-β-D-thiogalactopyranosid (IPTG) and then grown at 18 °C for 20 h. Bacteria were harvested by centrifugation and lysed through sonication. Recombinant protein was purified using immobilized metal affinity chromatography (IMAC) and the process monitored by SDS-PAGE. The size of the purified proteins was predicted using the online tool ExPASy (Gasteiger, 2003, Table 4). The molecular weight of the bands observed in SDS-PAGE were estimated using ThermoScientific PageRuler and the standard curve method. For Cas9-Doc1 the estimation by standard curve was slightly lower than the ExPASy prediction, while for Cip7-GFP and Cip7-RT it was the other way around (Table 4). The protein concentration in the elution fraction was determined by a Bradford assay.

TABLE 4
Molecular weights and isoelectric points of recombinant proteins.

| Protein | pl* | MW* [kDA] | MW** [KDa] |
|---|---|---|---|
| Cas9-Doc1 | 9.13 | 170 | 149 ± 20 |
| Cip7-GFP | 5.86 | 47 | 57 ± 9 |
| Cip7-RT | 7.95 | 94 | 109 ± 15 |

*Estimated by the online tool ExPASy (Gasteiger, 2003),
**Determined by SDS-PAGE standard curve method. The uncertainties were obtained through error propagation of the linear regression standard deviation.

The protein bands for Cas9-Doc1 and Cip7-GFP appeared very prominent in the elution fraction with a high contrast to the background, suggesting a high grade of purity. The Cip7-RT band, on the other hand, was more more faint, which decreased the contrast to the background. Note that for the SDS-PAGE the loaded amounts were not normalized to the culture volume. Cas9-Doc1 had an average protein yield of (22±11) mg/L, Cip7-GFP (94±31) mg/L and Cip7-RT (15±10) mg/L. Thus, Cip7-GFP had the greatest yield. Cas9-Doc1 and Cip7-RT are bigger in size (Table 4), which is why lower protein yield might be expected.

When Cip7-RT was grown without IPTG induction, the culture reached higher OD600 values and higher band intensity compared to the induced sample (data not shown). Impaired cell growth is expected upon induction with IPTG and was also observed for Cas9-Doc1 and Cip7-GFP, although not as severe. Basal expression of protein in the absence of IPTG could be seen for all three constructs (data not shown). However, for Cas9-Doc1 and Cip7-GFP the IPTG-induced samples the recombinant protein yield was much higher than the non-induced control, while Cip7-RT showed comparable and possibly even higher yield for non-induced samples against induced. Yields for the Cip7-RT non-induced were almost as high as Cas9-Doc1 induced, but the induced cultures yielded only about half of the protein amount.

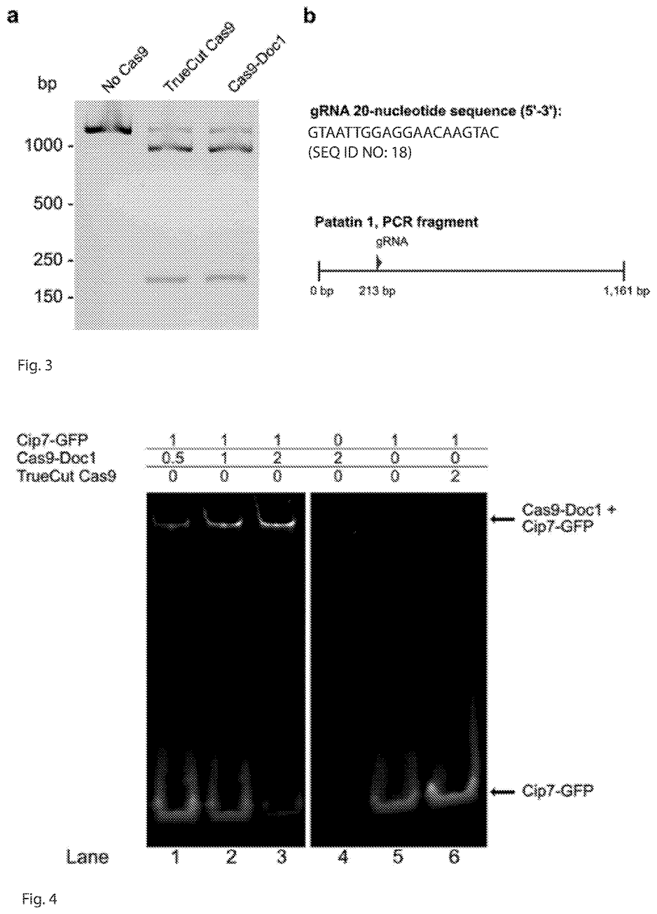

To investigate if the purified Cas9-Doc1 shows nuclease activity, an in-vitro cleavage assay was performed (FIG. 3A). A Patatin 1 PCR fragment was incubated either with commercial TrueCut Cas9 or purified Cas9-Doc1 and gRNA. The reactions were run on an agarose gel. As target DNA, the Patatin 1 gene was amplified using PCR. A previously validated gRNA was used to cleave the PCR fragment (FIG. 3B). A commercial Cas9 protein was used as control. Correct cleavage of the target DNA would produce two cleaved fragments with 948 bp and 213 bp in size, as observed. The assay clearly showed that the purified Cas9-Doc1 is active and cleaves efficiently the target DNA. There is no visible difference in the amount of cleaved DNA compared to the commercial Cas9. The assay was repeated after storing the purified Cas9-Doc1 for 14 days at −20 °C in reaction buffer with 50% glycerol and no decrease in activity could be observed.

### 2.3 Protein-Protein Interaction

The interaction of Cas9-Doc1 and Cip7-GFP was examined using native PAGE and in-gel GFP imaging (FIG. 4). Cip7-GFP and Cas9-Doc1 were loaded to a native gel, with increasing amounts of the latter one. TrueCut Cas9 was used as a negative control. Depicted values are in pmol. After electrophoresis the gel was imaged for GFP signal.

Specifically, Cas9-Doc1 was mixed with Cip7-GFP in increasing concentrations of the former one and incubated for 5 min at RT. A commercial Cas9 protein without Doc1 was used as a negative control. When just Cip7-GFP was loaded a single distinct band was observed (FIG. 4, lane 5). When adding Cas9-Doc1 a second band appeared above the first one, which became stronger with increasing amounts of Cas9-Doc1 while the lower band started to fade (FIG. 4, lane 1 to 3). The upper band could not be observed when Cip7-GFP was mixed with the commercial Cas9, that lacks the Doc1 domain (FIG. 4, lane 6). No bands could be observed in the lane with just Cas9-Doc1 (FIG. 4, lane 4). Note that the upper band is located immediately below the well of the gel and even extension of the running time did not make the upper band migrate further into the gel. Coomassie staining indicated a large amount of protein immediately below the well, at approximately the position, where the upper fluorescent band was observed (data not shown). The gradient in intensity of the upper band proportional to the amount of added Cas9-Doc1 shows interaction between Cas9-Doc1 and Cip7-GFP. At a ratio of 2:1 (Cas9-Doc1: Cip7-GFP) almost all Cip7-GFP was retained. These results showed that the recombinant proteins Cas9-Doc1 and Cip7-GFP bind to one another in an efficient and specific manner.

Previous studies on the expression and purification of cohesin and dockerin domains reported that the addition of CaCl₂ to the dockerin culture medium was required due to the Ca²⁺ dependent binding. However, under the conditions presented here no differences could be found in the interaction when the Cas9-Doc1 growth medium was supplemented with CaCl2 compared to no supplementation (data not shown).

To further verify the interaction between Cip7-GFP and Cas9-Doc1, in particular upon noting that Cas9 not entering the gel could pose a problem for definitive evidence of Cip7-GFP and Cas9-Doc1 interaction, the Cas9 in-vitro cleavage assay previously described was adapted into an electrophoretic mobility shift assay (EMSA) as inspired by Kunne et al., 2015. In short, Cas9 is guided by the gRNA and binds to the target DNA. By removing the final heating and protein degradation step in the cleavage assay and immediate cooling on ice we sought to keep Cas9 associated with the target DNA. The principal idea was that the negative charge of the target DNA would be sufficient to let DNA and the associated Cas9 protein migrate into the gel. The additional mass of Cas9 should cause a mobility shift, distinguishable from unbound DNA. When Cip7-GFP is added, a fluorescent band could be observable at the position of that mobility shift. Furthermore, the extra mass of Cip7-GFP or Cip7-RT coupled to Cas9-Doc1 could cause an additional mobility shift, which would further confirm the interaction.

After several rounds of optimization, this idea translated into a comprehensive protocol. After preincubation of the recombinant proteins, the target DNA was added and the reactions incubated at 37 °C for 30 min. This incubation time is reduced compared to the original cleavage assay to let time for binding but not complete cleavage.

After incubation the reactions were cooled on ice and run on an agarose gel without any DNA dye at low voltage. The gel was imaged for GFP and subsequently post-stained with GelRed for DNA visualization.

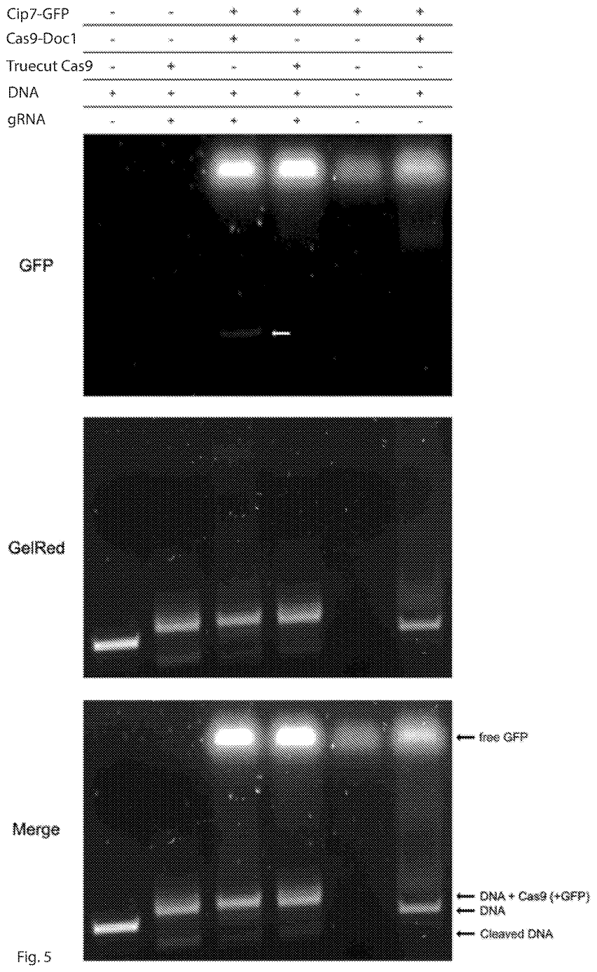

In samples with Cas9 and gRNA, a DNA band could be observed that appeared to be higher than the band corresponding to the uncleaved PCR fragment (FIG. 5, GelRed image). The band could not be observed when just the DNA was loaded (lane 1) or when gRNA was omitted (lane 6). This showed that binding of Cas9 to the DNA caused a mobility shift as expected. A lower DNA band most likely corresponding to the cleaved DNA fragment was observed when all three DNA, Cas9 and gRNA were present. Note that the second cleaved DNA fragment was located substantially lower as in FIG. 3, but is not shown in the image. When the gRNA was omitted (lane 6), no clear mobility shift band was observed. In all samples to which Cip7-GFP was added, a very prominent fluorescent band showed at the upper part of the gel. Solely Cip7-GFP (lane 5), gave a fluorescent band at the same position as the other samples, which is why this band can be attributed to free Cip7-GFP. A second distinct GFP band could only be observed when Cas9-Doc1+Cip7-GFP with gRNA was added to DNA (lane 3, GFP image, white arrow). This band appeared to be at the same position as the DNA mobility shift band, indicating an interaction of Cip7-GFP with Cas9-Doc1 (lane 3, Merge). No such GFP band could be observed when Cas9-Doc1 was replaced with the commercial Cas9 that lacks the Doc1 domain.

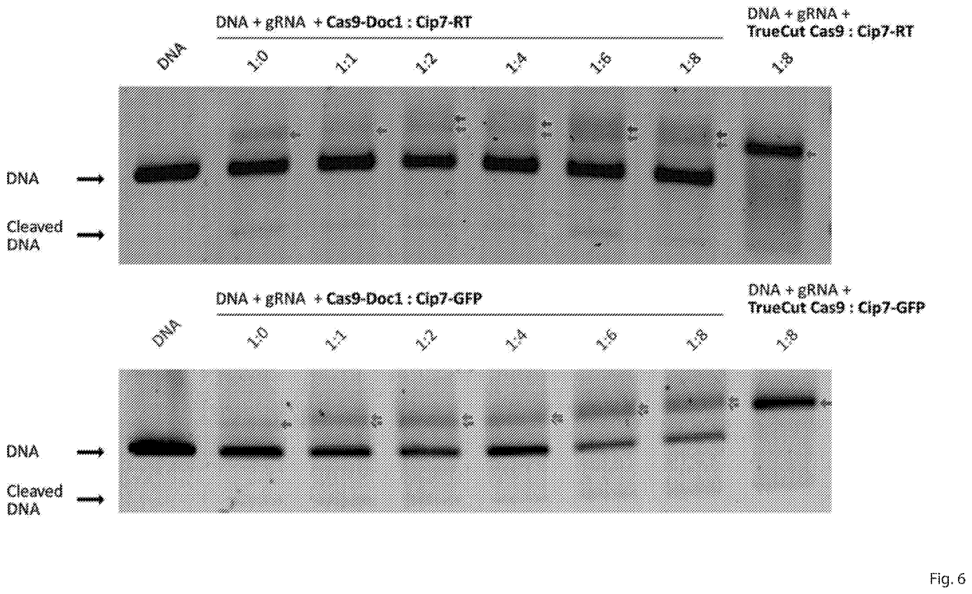

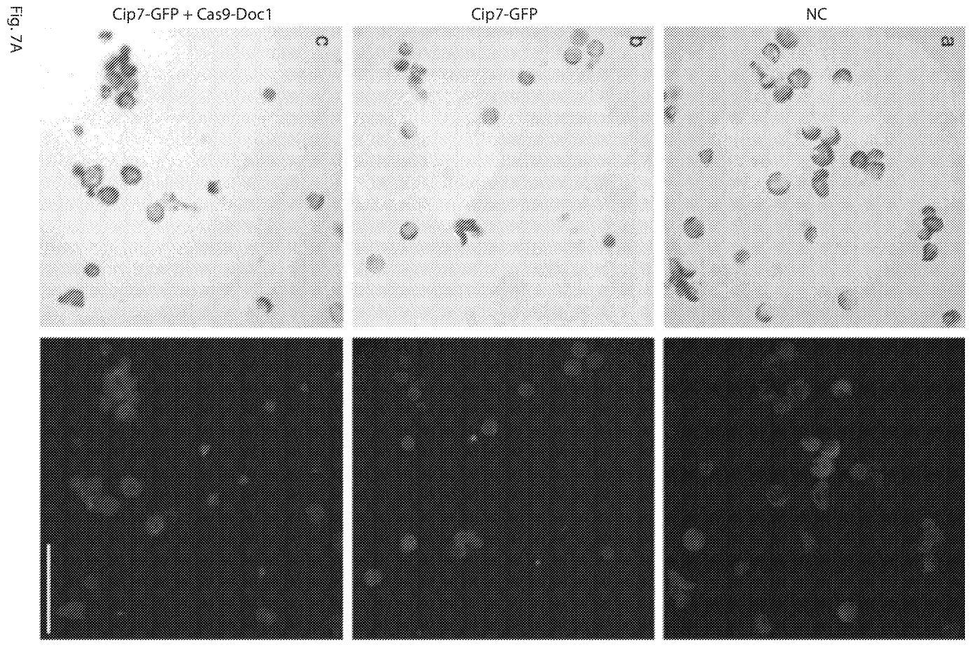

To investigate the interaction of Cip7-RT and Cas9-Doc1, the experiment was repeated with longer running time and overall reduced amounts of DNA and protein to achieve higher band resolution (FIG. 6). For comparison, Cip7-GFP and Cas9-Doc1 were included. The GFP imaging step was omitted, because the lower amounts of Cip7-GFP did not allow to observe the Cas9-Doc1+Cip7-GFP band as for FIG. 5 and for Cip7-RT and Cas9-Doc1 a GFP signal is not expected. The experimental procedure was kept the same but Cip7-RT or Cip7-GFP, respectively, were stepwise-increased this time. For the commercial Cas9 the mobility shift band (indicated with arrows) is much more prominent and the uncleaved DNA band has disappeared, while for all Cas9-Doc1 samples the uncleaved DNA band is much more prominent than the mobility shift band (FIG. 6). With increasing amounts of Cip7-RT the mobility shift band appears more diffuse and wider until it has about double the width of the initial band (FIG. 6, upper panel). This could not be observed for the control with the commercial Cas9. The wider band could be due to the predicted extra mobility shift caused by the additional mass of Cip7-RT when coupled to Cas9-Doc1. The lower part of the mobility shift band, does not fade with increasing amounts of Cip7-R. A similar situation emerges for Cip7-GFP. The mobility shift band appears to become wider with increasing Cip7-GFP amounts, even though not as much as for Cip7-RT. These results show specific interaction of Cas9 and the functional proteins through the adapter domains Doc1 and Cip7.

### 2.4 RNP Transfection

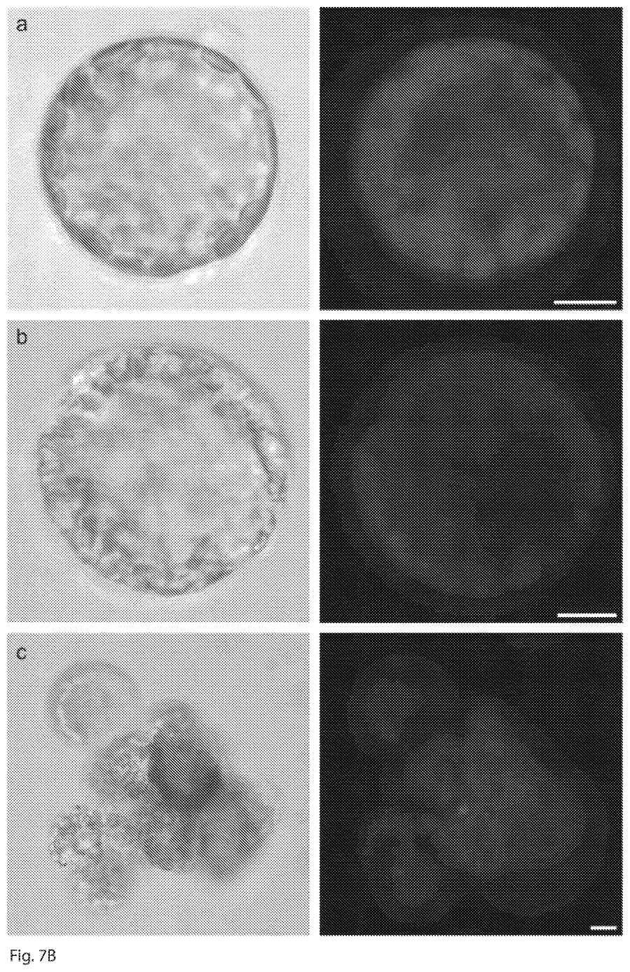

Potato (Solanum tuberosum) protoplasts were transfected with the purified fusion proteins to investigate if the interaction of Cas9-Doc1 and Cip7-GFP that was already observed in-vitro can also be seen in-vivo. Furthermore, there was a need to assess if transfected protoplasts retained cell division ability and thus the grade of purity for the fusion proteins is sufficient. For this, protoplasts were isolated from potato leaves and transfected with 40% PEG. Protoplasts transfected with Cip7-GFP, Cip7-GFP+Cas9-Doc1 and the negative control without transfected protein were imaged using fluorescence microscopy with UV as light source (FIGS. 7A and 7B). Cas9-Doc1 has a NLS upstream of the SpCas9 gene which signals import into the nucleus. If Cip7-GFP couples to Cas9-Doc1 the complex might be imported and a fluorescence signal in the nucleus observed. Chloroplasts are the light harnessing organelles of plant cells and carry a pigment called chlorophyll, which has an emission maximum at about 730 nm when excited with UV light. Emitted light at that wavelength is perceived by the human eye as red light. The GFP variant GFPuv has an emission maximum at about 506 nm, which is perceived as green light. Note that bandpass filters for autofluorescence and GFP signal were not available during the experiment, which is why both can only be distinguished by the recorded color in the UV image. Generally, the negative control seemed to display slightly more intact protoplasts than the samples that were transfected with the recombinant proteins (FIG. 7A, left panel, images representative for multiple observations). In the negative control a strong autofluorescence signal from chloroplasts could be seen in the spectrum of red light, and as expected, green light that would indicate GFP signal could not be observed (FIG. 7A, a). Cip7-GFP together with the autofluorescence, showed several green spots that indicates GFP signal (FIG. 7A, b). Cip7-GFP+Cas9-Doc1 seemed to have a greater amount of GFP signal compared to just Cip7-GFP (FIG. 7A, c).

Protoplasts transfected with RNPs and functional proteins, were embedded in alginate and allowed to divide over two weeks. The number of calli was counted as a measure of cell division ability, to evaluate if the purity of the purified recombinant proteins is sufficient for protoplast transfection and regeneration. High statistical significance in the difference for the number of calli was present in NC compared to the other samples with transfected proteins. Among the samples transfected with recombinant proteins no statistical significance could be observed. Interestingly, protoplasts transfected with only Cas9-Doc1 seemed to show a slightly higher cell division ability with 4 calli per 50 000 pps compared to the sample with Cas9-Doc1 and gRNA that had about 2 calli per 50 000 pps. Protoplasts transfected with Cip7-GFP in addition to Cas9-Doc1 and gRNA did not show further impairment of regeneration having about 2 calli per 50 000 pps.

### 3. Discussion

Here, we present the foundation for a modularly interactive CRISPR system, that separates the fusion proteins into plug-and-play components, enabling both higher flexibility and efficient protein production in bacterial expression systems.

### 3.1 Protein Expression

Even though the molecular weights predicted by ExPASy did not match exactly the ones determined by SDS-PAGE (Table 4), the closeness suggests that the bands represent the recombinant proteins, since some variation is expected when using the SDS-PAGE standard curve method (Karpe and Hamsten, 1994). It can be assumed that both Cas9-Doc1 and Cip7-GFP were successfully expressed and purified using the T7 expression system in Rosetta (DE3) E. coli cells. A high grade of purity could be achieved, which was assessed by SDS-PAGE. For Cas9-Doc1 the average yield of (22±11) mg/L is in line with reports from Fleitas et al., 2022 for SpCas9 fusion proteins. The Cas9 in-vitro cleavage assay showed that the purified Cas9-Doc1 is active and no difference in activity compared to the commercial Cas9 could be seen. Thus, the dockerin domain Doc1 does not impair the nuclease activity of Cas9.

For Cip7-RT the non-induced protein yield reached almost the amounts of induced Cas9-Doc1 and the induced yield was only half of that. The poor yield for induced Cip7-RT cultures might indicate toxicity of the protein in high amounts and marks the necessity for further optimization of the culture conditions. Culture medium free of expression-inducing reagents as well as different temperatures and growth times might provide better yield for Cip7-RT. Furthermore, the coexpression of T7 lysozyme has earlier been shown to give tighter control over protein expression (Spehr et al., 2000). T7 lysozyme can inhibit the T7 polymerase when the latter one is basally expressed, which allows for tighter control of and at the same time enables easier cell lysis using freeze-thaw cycles (Wanarska et al., 2007).

All in all, the possibility of easy protein production in the modular CRISPR-Cas system is an advantage to current approaches that need either extensive optimization procedures or employ eukaryotic expression systems with much higher costs.

### 3.2 Protein-Protein Interaction Studies

The native PAGE with in-gel GFP imaging effectively showed interaction between Cas9-Doc1 and Cip7-GFP (FIG. 4). Interaction between Cas9-Doc1 and Cip7-GFP was then confirmed using an EMSA (FIG. 5). A GFP signal could be observed at the position of the mobility shift band only in the sample with Cas9-Doc1 and Cip7-GFP (8, lane 3). This shows interaction of Cas9-Doc1 and Cip7-GFP also when the former one binds to DNA. Furthermore, in the absence of gRNA no mobility shift of the DNA band could be observed, demonstrating the specificity of the purified Cas9-Doc1 (FIG. 5, lane 5). The repetition of the assay with longer running time to visualize the additional band shift when Cip7-GFP or Cip7-RT are added, confirmed the interaction already observed for Cas9-Doc1 and Cip7-GFP also for Cas9-Doc1 and Cip7-RT (FIG. 6).

The addition of Cip7-RT seemed to cause a stronger mobility shift than the addition of Cip7-GFP demonstrated by the band width. This could be due to the smaller size of Cip7-GFP that is only about half of Cip7-RT (Table 4).

### 3.3 Protoplast Transfection

Potato protoplasts transfected with Cip7-GFP displayed distinct spots of green fluorescence, indicating that a sufficient amount of recombinant protein was used for transfection (FIG. 7A).

Protoplasts transfected with recombinant proteins clearly showed a reduced survivability compared to protoplasts treated with only 40% PEG. This likely demonstrates the stress that the procedure poses to the cells. Even though not significant, there seemed to be a tendency of the commercial TrueCut Cas9 achieving a higher cell division ability than the purified Cas9-Doc1. This could indicate higher purity of the commercial Cas9-Doc1 leading to better cell division ability, pointing to an advantage of further purification steps. One plausible reason for this observation could be traces of bacterial compounds in the purified protein samples that cause an increased immune response by the protoplasts where pathogens triggered the immune response of protoplasts and induced cell death. Rajagopalan et al., 2018 described a two-step method for obtaining highly pure Cas9 protein, involving a cation exchange chromatography step after the IMAC purification. The additional step might achieve higher purity and therefore achieve better protoplast regeneration, and that two-step method could be used when producing the CRISPR-Cas protein.

Cas9-Doc1 without gRNA seemed to achieve a slightly higher cell division ability than the samples with gRNA, possibly indicating cell stress by the nuclease activity. The addition of Cip7-GFP to Cas9-Doc1 and gRNA on the other hand did not display any further impairment of cell division.

### 3.4 Conclusion

This example showed that the modularly interactive CRISPR system can expand the molecular toolbox for RNP delivery methods. The expression and purification of the first components, i.e. the Cas9 protein with its dockerin binding peptide (Cas9-Doc1) and the functional protein (reverse transferase with its cohesion binding peptide (Cip7-RT) was successful and will pave the way for further advancements of the system. The components can be produced in large scale in bacterial cells and thereby provide a better cost efficiency and thus increase the accessibility to CRISPR-Cas gene editing. The results further show that Cas9-Doc1 couples with Cip7-GFP and Cip7-RT to complexes as intended, whereby Cas9 retains the ability to bind and cleave DNA. Protoplasts could be transfected with the recombinant proteins and results indicate successful cell division. By freely combining the different components a higher flexibility is achieved. This modular system holds great promise in inter alia changing the way genome editing shapes sustainable food production.

### REFERENCES

Andersson, M., Turesson, H., Olsson, N., Falt, A.-S., Ohlsson, P., Gonzalez, M. N., Samuelsson, M., & Hofvander, P. (2018). Genome editing in potato via CRISPR-cas9 ribonucleoprotein delivery. Physiologia Plantarum, 164(4), 378-384. https://doi.org/10. 1111/ppl.12731
Fleitas, A. L., Señorale, M., & Vidal, S. (2022). A robust expression and purification method for production of SpCas9-GFP-MBP fusion protein for in vitro applications. Methods and Protocols, 5(3), 44. https://doi.org/10.3390/mps5030044
Gasteiger, E. (2003). ExPASy: The proteomics server for in-depth protein knowledge and analysis. Nucleic Acids Research, 31(13), 3784-3788. https://doi.org/i0.1093/nar/gkg563
Karpe, F., & Hamsten, A. (1994). Determination of apolipoproteins b-48 and b-100 in triglyceride-rich lipoproteins by analytical SDS-PAGE. Journal of Lipid Research, 35(7), 1311-1317. https://doi.org/10.1016/s0022-2275(20)39974-0
Künne, T., Westra, E. R., & Brouns, S. J. J. (2015). Electrophoretic mobility shift assay of DNA and CRISPR-cas ribonucleoprotein complexes. Methods in molecular biology (pp. 171-184). Springer New York. https://doi.org/10.1007/978-1-4939-2687-9_11
New England Biolabs. (2014). In vitro digestion of dna with cas9 nuclease, S. pyogenes (m0386). protocols.io. https://doi.org/dx.doi.org/10.17504/protocols.io.ch2t8d
R Core Team. (2022). R: A language and environment for statistical computing. R Foundation for Statistical Computing. Vienna, Austria. https://www.R-project.org/
Rajagopalan, N., Kagale, S., Bhowmik, P., & Song, H. (2018). A two-step method for obtaining highly pure cas9 nuclease for genome editing, biophysical, and structural studies. Methods and Protocols, 1(2), 17. https://doi.org/10.3390/mps1020017
Rees, H. A., Komor, A. C., Yeh, W.-H., Caetano-Lopes, J., Warman, M., Edge, A. S. B., & Liu, D. R. (2017). Improving the DNA specificity and applicability of base editing through protein engineering and protein delivery. Nature Communications, 8(1). https: //doi.org/10.1038/ncomms15790
Spehr, V., Frahm, D., & Meyer, T. F. (2000). Improvement of the t7 expression system by the use of t7 lysozyme. Gene, 257(2), 259-267. https://doi.org/10.1016/s0378-1119(00)00400-5
Wanarska, M., Hildebrandt, P., & Kur, J. (2007). A freeze-thaw method for disintegration of Escherichia coli cells producing t7 lysozyme used in pbad expression systems. Acta biochimica Polonica, 54, 671-672.

1-15: (canceled)

16. A modular CRISPR-Cas system for ribonucleoprotein (RNP) delivery comprising:
a CRISPR-Cas protein fused to a first binding peptide, and
a functional protein fused to a second binding peptide,
wherein the first binding peptide and the second binding peptide are configured to bind to each other and define a binding peptide pair.

17. The modular CRISPR-Cas system according to claim 16, wherein the functional protein is configured to modify the outcome of the action of the CRISPR-Cas protein on DNA.

18. The modular CRISPR-Cas system according to claim 16, wherein the binding peptide pair comprises a Cohesin/Dockerin system.

19. The modular CRISPR-Cas system according to claim 16, wherein the functional protein is selected from:
a) deaminases,
b) reverse transcriptases,
c) methylases, and
d) homologous recombination and homology directed repair enhancing proteins.

20. The modular CRISPR-Cas system according to claim 16, further comprising a guide RNA sequence complex bound to the CRISPR-Cas protein.

21. The modular CRISPR-Cas system according to claim 16, further comprising:
a first container containing the CRISPR-Cas protein fused to the first binding peptide, and
a second container containing the functional protein fused to the second binding peptide.

22. The modular CRISPR-Cas system according to claim 16, wherein the CRISPR-Cas protein fused to the first binding peptide, and the functional protein fused to the second binding peptide, are provided in separate buffer solutions.

23. The modular CRISPR-Cas system according to claim 16, wherein the CRISPR-Cas protein fused to the first binding peptide, and the functional protein fused to the second binding peptide, are provided in solid form.

24. The modular CRISPR-Cas system according to claim 16, further comprising a further functional protein fused to a third binding peptide, wherein the further functional protein is different from the functional protein, and wherein the third binding peptide is configured to bind to the first binding peptide.

25. The modular CRISPR-Cas system according to claim 24, wherein the third binding peptide has the same amino acid sequence as the second binding peptide,

26. The modular CRISPR-Cas system according to claim 24, wherein the further functional protein fused to the third binding peptide is provided in a separate buffer solution or provided in a solid form.

27. A method of assembling a CRISPR-Cas-complex for ribonucleoprotein (RNP) delivery comprising the steps of:
i. providing the system according to claim 16; and
ii. contacting the CRISPR Cas protein fused to the first binding peptide with the functional protein fused to the second binding peptide.

28. The method of assembling a CRISPR-Cas-complex for ribonucleoprotein (RNP) delivery according to claim 27, wherein the method further comprises contacting the CRISPR Cas protein with a guide RNA sequence.

29. The method according to claim 27, wherein step ii comprises mixing the CRISPR Cas protein fused to the first binding peptide with the functional protein fused to the second binding peptide.

30. The method according to claim 27, wherein the CRISPR-Cas protein with its first binding peptide and the functional protein with its second binding peptide are provided in separate buffer solutions, such that the buffer solutions can be mixed to assemble the CRISPR-Cas-complex.

31. A CRISPR-Cas complex for ribonucleoprotein (RNP) delivery comprising:
a CRISPR-Cas protein fused to a first binding peptide, and
a functional protein fused to a second binding peptide,
wherein the first binding peptide and the second binding peptide are bound to each other.

32. The CRISPR-Cas complex for ribonucleoprotein (RNP) delivery according to claim 31, wherein the CRISPR-Cas complex further comprises a guide RNA sequence complex bound to the CRISPR-Cas protein.

33. Use of the modular CRISPR-Cas system for ribonucleoprotein (RNP) delivery according to claim 16, for modifying the genome of a plant selected from maize, rice, sorghum, rye, barley, wheat, millet, oats, sugarcane, turfgrass, switchgrass, soybean, canola, alfalfa, sunflower, cotton, tobacco, peanut, potato, sugar beet, grape, Arabidopsis or safflower cell.

34. The use of the modular CRISPR-Cas system for ribonucleoprotein (RNP) delivery according to claim 33, wherein the plant is from the family Solanaceae, and the genus Solanum.

35. The use of the modular CRISPR-Cas system for ribonucleoprotein (RNP) delivery according to claim 33, wherein the plant is from the species Solanum tuberosum.


SEQUENCE LISTING (23 sequences)

SEQ ID NO: 1 - DNA, 67 bp, synthetic construct
  source 1..67: mol_type = other DNA
ctagatttgtttaactttaagaaggagatataccatgcccaagaagaagaggaaggtgactagtcca

SEQ ID NO: 2 - DNA, 67 bp, synthetic construct
  source 1..67: mol_type = other DNA
gatctggactagtcaccttcctcttcttcttgggcatggtatatctccttcttaaagttaaacaaat

SEQ ID NO: 3 - DNA, 46 bp, synthetic construct
  source 1..46: mol_type = other DNA
ctagatttgtttaactttaagaaggagatataccatgactagtcca

SEQ ID NO: 4 - DNA, 46 bp, synthetic construct
  source 1..46: mol_type = other DNA
gatctggactagtcatggtatatctccttcttaaagttaaacaaat

SEQ ID NO: 5 - DNA, 18 bp, synthetic construct
  source 1..18: mol_type = other DNA
ggcgtagaggatcgagat

SEQ ID NO: 6 - DNA, 18 bp, synthetic construct
  source 1..18: mol_type = other DNA
caagtggagtagatgcta

SEQ ID NO: 7 - DNA, 18 bp, synthetic construct
  source 1..18: mol_type = other DNA
tgcatggatgactcggaa

SEQ ID NO: 8 - DNA, 19 bp, synthetic construct
  source 1..19: mol_type = other DNA
gacaaggcgatagtttaca

SEQ ID NO: 9 - DNA, 20 bp, synthetic construct
  source 1..20: mol_type = other DNA
aaactcgccaaatcactaag

SEQ ID NO: 10 - DNA, 20 bp, synthetic construct
  source 1..20: mol_type = other DNA
cgttaaagagttactaggga

SEQ ID NO: 11 - DNA, 18 bp, synthetic construct
  source 1..18: mol_type = other DNA
ggcgtagaggatcgagat

SEQ ID NO: 12 - DNA, 20 bp, synthetic construct
  source 1..20: mol_type = other DNA
cacttgtcactactttctct

SEQ ID NO: 13 - DNA, 19 bp, synthetic construct
  source 1..19: mol_type = other DNA
acgaaaccagcaaagaacc

SEQ ID NO: 14 - DNA, 20 bp, synthetic construct
  source 1..20: mol_type = other DNA
tttcaaaaactcacctaccc

SEQ ID NO: 15 - DNA, 19 bp, synthetic construct
  source 1..19: mol_type = other DNA
ggtagaagccctggtgaaa

SEQ ID NO: 16 - DNA, 26 bp, synthetic construct
  source 1..26: mol_type = other DNA
atggcaactactaaatcttttttaat

SEQ ID NO: 17 - DNA, 25 bp, synthetic construct
  source 1..25: mol_type = other DNA
ttaataagaagctttgtttgctcgg

SEQ ID NO: 18 - DNA, 20 bp, synthetic construct
  source 1..20: mol_type = other DNA
gtaattggaggaacaagtac

SEQ ID NO: 19 - protein, 144 aa, synthetic construct
MAVRIKVDTVNAKPGDTVRIPVRFSGIPSKGIANCDFVYSYDPNVLEIIEIEPGELIVDPNPTKSFDTAVYPDRKMIVFLFAEDSGTGAYAITEDGVFATIVAKVKSGAPNGLSVIKFVEVGGFANNDLVEQKTQFFDGGVNVG

SEQ ID NO: 20 - DNA, 489 bp, synthetic construct
  source 1..489: mol_type = other DNA
acgccagatctgcaccaccaccaccaccacgcggtgcgcattaaagtggataccgtgaacgcgaaaccgggcgataccgtgcgcattccggtgcgctttagcggcattccgagcaaaggcattgcgaactgcgattttgtgtatagctatgatccgaacgtgctggaaattattgaaattgaaccgggcgaactgattgtggatccgaacccgaccaaaagctttgataccgcggtgtatccggatcgcaaaatgattgtgtttctgtttgcggaagatagcggcaccggcgcgtatgcgattaccgaagatggcgtgtttgcgaccattgtggcgaaagtgaaaagcggcgcgccgaacggcctgagcgtgattaaatttgtggaagtgggcggctttgcgaacaacgatctggtggaacagaaaacccagttttttgatggcggcgtgaacgtgggcggtggttctggtcatatgggtaccacccag

SEQ ID NO: 21 - protein, 70 aa, synthetic construct
MPPVLLGDVNGDGTINSTDLTMLKRSVLRAITLTDDAKARADVDKNGSINSTDVLLLSRYLLRVIDKFPV

SEQ ID NO: 22 - DNA, 406 bp, synthetic construct
  source 1..406: mol_type = other DNA
tcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacggtggttctggtccgccggtgctgctgggcgatgtgaacggcgatggcaccattaacagcaccgatctgaccatgctgaaacgcagcgtgctgcgcgcgattaccctgaccgatgatgcgaaagcgcgcgcggatgtggataaaaacggcagcattaacagcaccgatgtgctgctgctgagccgctatctgctgcgcgtgattgataaatttccggtgcccaagaagaagaggaaggtgctcgagcaccac

SEQ ID NO: 23 - protein, 6 aa, synthetic construct
HHHHHH